(19) United States
(12) Patent Application Publication
(10) Pub. No.: US 2007/0292839 A1
(43) Pub. Date: Dec. 20, 2007

(54) Assay

(76) Inventor: David Parker, Kent (GB)

Correspondence Address: MORGAN LEWIS & BOCKIUS LLP, 1111 PENNSYLVANIA AVENUE NW, WASHINGTON, DC, 20004, US

(21) Appl. No.: 10/577,358

(22) PCT Filed: Nov. 1, 2004
(86) PCT No.: PCT/GB04/04621

§ 371 Date: Apr. 16, 2007

(30) Foreign Application Priority Data
Oct. 31, 2003 (GB) 0325483.6

(51) Int. Cl. C12Q 1/70; G01N 33/556

(52) U.S. Cl. 435/5; 436/501

(57) ABSTRACT

The invention relates to a method of determining the amount or presence of antibodies to an immunogen in a blood sample comprising at least the steps of detecting the amount or presence of plasma antibodies or parts thereof and lymphocyte antibodies or parts thereof in said sample, wherein said plasma and lymphocyte antibodies are detected together or in separate assays and determination of their combined amount or presence determines the amount or presence of antibodies to said immunogen. Uses of the method in high throughput screening, screening of blood bank samples and in diagnosis are also provided.

This invention is in the field of antibody detection, and in particular relates to the detection of antibodies in blood samples produced in response to antigen challenge such as by infection or vaccination etc., by means of a simple method involving determining the presence of both lymphocyte and plasma antibodies in the same blood sample. Preferably, the plasma and lymphocyte antibodies are assessed simultaneously in a single assay.

There are several techniques available to detect antibody levels in samples, for example the ELISA test. Most commonly, ELISA is used as a serological assay, but it is also used to study the immunochemical properties of antigens or antibodies, and has frequently found application in, for example, the evaluation and characterisation of immune responses, to investigate antibody production by cell cultures, in hybridoma technology etc.

The ELISA assay is simple to use, sensitive and relatively quick, but it is only able to measure the presence of secreted target antibodies in the sample. It cannot distinguish between on-going antibody synthesis in response to the antigen, and antibodies already present from past infection, or by passive transfer etc. Only antibodies that have been secreted from lymphocytes are detected. Lymphocyte antibodies that have not been secreted are not detected in this technique.

In addition to the ELISA test, the ELISPOT or enzyme linked immunospot assay has been used, as reviewed for example by Czerkinsky et al. in ELISA and other Solid Phase Immunoassays, Eds. D. M. Kemeny and S. J. Challacombe, 1988, Chapter 10, 217-239. This technique, based on the ELISA method, enables the enumeration of lymphocytes secreting antibodies against one or more target antigens. Basically, the ELISPOT is a variant of the ELISA method, whereby antibody secreting cells (ASC) may be revealed by culturing lymphocytes in specially modified ELISA wells coated with the target antigen, and by replacing the standard ELISA reagents with enzyme-substrate complexes that yield a coloured precipitate (spots), adjacent to the secreting cell. Spots can then be counted to give a measure of the number of antibody-producing cells. Protein synthesis inhibitors may be included in the culture medium, to confirm that the spots detected are due to de novo antibody synthesis, during the in vitro incubation period.

Whilst the ELISPOT technique has proved very useful in studying the dynamics of humoral immune responses, and has been used to detect spontaneous ASC that appear transiently in the peripheral circulation of immunised subjects, certain features of the method place constraints on its use in a clinical diagnostic setting. Firstly, since individual spots need to be counted for each sample, which can be time consuming and laborious, the method is not particularly suited to the analysis of large numbers of samples, such as occurs in a clinical diagnostic laboratory. Secondly, only the number of cells secreting antibodies in each sample is enumerated and generally speaking, this requires reasonably large sample volumes, e.g. several mls. ELISPOT plates are also expensive and the assay is not readily amenable to automation.

More recently, assays have been developed to detect on-going antibody synthesis. WO 96/26443, which is incorporated herein by reference, describes such an assay in which lymphocytes are cultured after isolation and the levels of antibodies produced and secreted from lymphocytes during that culture period are determined.

This technique thus necessarily requires incubation of the lymphocytes of the test sample at about 37 C in order to allow measurement of the antibodies secreted during incubation. The average incubation period is 2-5 hours which represents a significant limitation on the speed of performing the assay. Incubation also requires the provision of suitable equipment at the site of testing so that it may be carried out immediately prior to the assay procedure. This generally means that assay of the lymphocytes needs to be carried out shortly after a sample has been taken from a subject, because storage of samples, e.g. by freezing, is not acceptable due to the resulting decrease in cell viability. Purified cells can only be maintained viable for relatively short periods of time by storage on ice at 0 C or less favourably at 4 C.

WO 00/77525, which is incorporated herein by reference, describes a further antibody assay. This assay is specifically designed to detect lymphocyte contained antibodies prior to their secretion. In this assay, lymphocytes of the sample are lysed, and the antibodies that are released from the lymphocytes by this lysis are detected. This assay is based on the finding that lymphocyte disruption can yield sufficient quantities of “newly synthesised antibodies” to allow detection for immunodiagnostic purposes. Conveniently, in this method the lymphocytes are purified from the sample before lysis.

The assay described in WO 00/77525 therefore does not require the sample to be incubated in vitro for the synthesis of sufficient antibodies for detection purposes, as was the case with standard antibody detection assays which detect antibodies which have been secreted in vitro or in vivo. The assay is therefore of particular use in detecting acute infection in time periods shortly after challenge such as by infection or immunisation, when lymphocytes are rapidly producing antibodies in response to the challenge.

The method described in WO 00/77525 however only allows the detection of infection whilst active antibody production occurs, i.e. during a short time period following challenge. This window corresponds to that period of time during which cells are producing detectable levels of antibodies. After challenge, B lymphocytes start to synthesise antibody and divide in response to the antigen challenge. During this period of time, usually 3 to 10 days from infection, the lymphocytes rapidly synthesise the antibodies of interest and thus antibody levels in these cells are high. These cells are however short-lived and as a consequence, from around two weeks after infection the amount of relevant antibody being produced by the lymphocytes falls significantly. By approximately 2 weeks from infection, the amount of the relevant antibodies that are present in lysed lymphocytes has returned to low levels. This is shown, for example, in the influenza studies shown in WO 00/77525. Thus, this method, whilst very useful for detecting acute infection, will not detect on-going or past infection and is thus only of use in a relatively short time frame following challenge such as by infection or immunisation.

It will be appreciated that methods that allow the rapid and accurate detection of infection in samples from patients are required, and that there are significant advantages associated with the early detection of infection. Whilst there are techniques that allow the detection of acute infection (e.g. WO 00/77525) and techniques that allow the detection of past or on-going infection, no single test exists that is able to provide a rapid answer to the question of whether an individual has at some point in their lifetime been exposed to a particular challenge, e.g. infection i.e. have an acute, on-going (chronic) or past infection. It would therefore be extremely valuable to be able to identify whether challenge has occurred without requiring knowledge of the time of challenge. That same test could then be used for analysis of samples regardless of the time of challenge, e.g. for both chronic and acute infections. No single test currently addresses these needs.

Such tests would have particular utility in high-throughput screening in which the information on the infection status of the source organism is required. For example, blood banks require a rapid and accurate test to determine the safety of blood to be used for transfusion. This is particularly important in areas of the world with a high prevalence of blood-borne infectious disease, such as HIV and hepatitis, that can be passed on to recipients of the blood.

Current methods of detecting HIV infection and management of the risk of HIV transmission via blood transfusion are in place in certain areas. For example, a Blood Safety Policy has been implemented in South Africa and individuals at high risk of infection have questionnaires, donor education and one to one interview facilities at their disposal. These provisions, and self exclusion have all helped to significantly reduce the seroprevalence in this group of the population. This contrasts with the escalation of the HIV/AIDS pandemic in the general population.

The high HIV seroprevalence among prospective blood donors increases the risk of transmission of HIV in the immunosilent “window period” of infectivity. In 1994 this risk was estimated to be 2.2 infections per 100,000 donations during this window period in South Africa (Sitas F et al. S. Afr. Med. J. 1994; 84: 142-144). During this immunosilent window period it is not possible to detect HIV infection using known commercially available assays.

The implementation of the structured risk management programme has reduced this risk to recipients of blood products to about 1 in 100,000 donations as estimated by a standard mathematical model. The observed risk is about four times lower, but this may be because of factors making the diagnosis of transfusion-transmitted disease difficult.

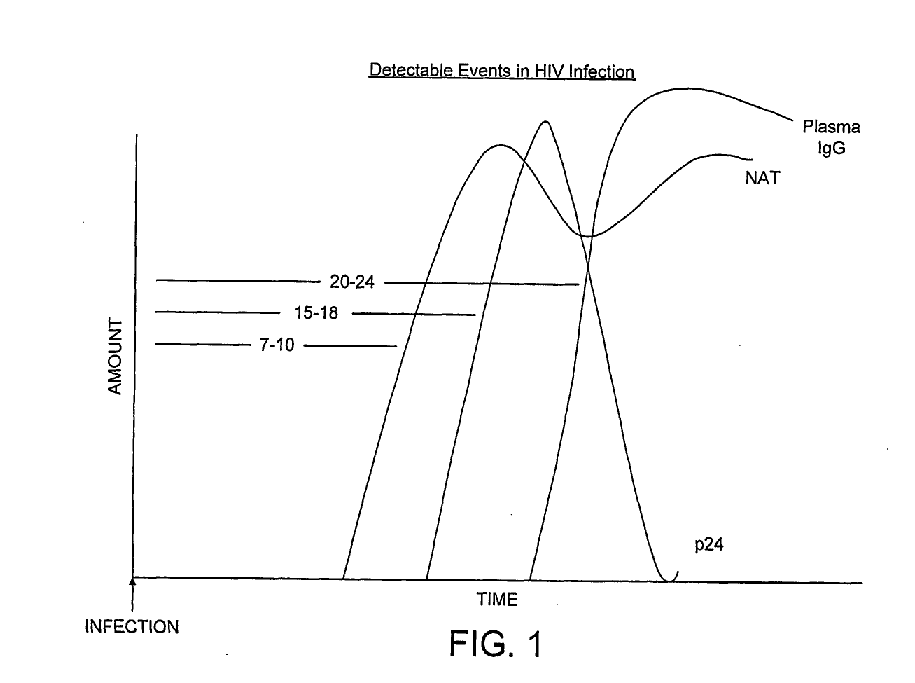

There are certain HIV tests currently available. For example the introduction of an HIVI p24 antigen test has been shown by studies in developed countries to shorten the immunosilent window period. The p24 assay detects infection at 15-18 days after infection (FIG. 1).

It has been considered that more sensitive tests, to detect plasma-derived viral nucleic acid in individual donations, may be the most effective way to decrease even further this window of infectivity. This is possible with Nucleic Acid Testing (NAT), but this technique has logistical problems of contamination, training, laboratory space, quality assurance and particularly cost. These factors have limited the introduction of NAT to a few centers in the more affluent countries (Busch M. P.: Chapter 2; Blood Safety in the New Millennium; Stramer S L Ed; AABB, 2001). The nucleic acid test detects infection at around 7 to 10 days post infection (FIG. 1).

The detection of IgG in plasma is also used to diagnose HIV, however IgG does not appear in the plasma at levels detectable by conventional assays until 20-24 days after infection as a result of accumulation following the intense period of production and secretion during the 2 weeks following infection (FIG. 1).

There is thus a 7-10 day period, immediately following infection, in which it is currently not possible to detect HIV infection using commercially available tests, particularly using tests that are suitable for use on a routine basis on large sample numbers (FIG. 1).

Furthermore, owing to the different timescales of the immunological events on which these various assays depend, multiple assays would need to be performed in parallel to be certain of detecting infection. For example, it can be seen from FIG. 1 that a negative result from simply performing a p24 test would only eliminate the presence of infection in a certain stage of the disease. Similarly, a nucleic acid test performed too early (i.e. before 7 to 10 days after infection) would not detect infection. Owing to the fact that in reality it is not known whether an individual is infected, let alone the specific date on which they became infected, these test show significant deficiencies.

The timescales indicated above relate to HIV infection, antibody production and detection. In the case of other challenges, this timing may vary, although an essentially parallel cycle is expected particularly for chronic infections. It will however be appreciated that as a result of the underlying immune processes, accumulation of antibodies to the challenge immunogen in detectable levels in plasma will inevitably lag behind antibody production in the lymphocytes and production of other markers of challenge by the immunogen are likely to have yet different timescales at which detection is possible. As such, different assays would be appropriate depending on the time elapsed since the challenge.

The present invention addresses these deficiencies and provides a single test that can identify infection as early as possible post-infection, without excluding the possibility of detection of infection at later stages.

Surprisingly it has now been found that a single test can be used to determine whether a mammal has been infected, e.g. at any time from approximately 3-7 days after infection. In this test an antibody assay is performed to determine the presence of antibodies in both the plasma (i.e. antibodies that have been secreted) and lymphocytes derived from a single blood sample. The assay is performed using both the plasma and lymphocyte portions of the sample, and the presence of either relevant lymphocyte antibodies or plasma antibodies or both gives a positive result. The period of time defined by the immunosilent ‘window of infection’ described above for HIV is therefore shorter using this technique than for any other known technique, and the ability to detect antibodies does not diminish once the antibodies in the lymphocytes have been secreted and are no longer detectable in the lymphocytes. The method thus allows detection e.g. less than 20, e.g. less than 15 or 10 days e.g. from 3 or 7 to 10 days post infection.

Particularly significantly, in view of the combination of results from lymphocyte and plasma antibodies, periods post-infection which have low levels of lymphocyte and/or plasma antibodies may be compensated and enhanced by the contribution of the other antibody type (e.g. at around 3 weeks post-infection). This is particularly advantageous when antibody levels are at the limits of detection and combination of the two antibody types can produce a detectable signal. Thus, methods in which the relevant portions of the samples are combined and assayed together are particularly preferred as discussed hereinafter. This allows analysis to be conducted from as early as a few days after infection (in which predominantly newly synthesised antibody would be detected), to several weeks after infection (when the predominant antibody would be the plasma antibody) until those plasma antibody levels decrease (approximately 12 to 15 months post-production in cases in which the infectious agent is eradicated from the organism). In the cases of chronic infection in which the challenge remains, e.g. HIV or hepatitis infection, the assay can continue to be conducted. Thus it is possible to detect either lymphocyte antibodies or plasma antibodies at any time from e.g. 3-7 days after infection.

In one aspect therefore, the invention provides a method of determining the amount or presence of antibodies to an immunogen in a blood sample comprising at least the steps of:

obtaining a blood sample (comprising plasma and lymphocytes); and

detecting the amount or presence of plasma antibodies or parts thereof and lymphocyte antibodies or parts thereof in said sample;

wherein said plasma and lymphocyte antibodies are detected together or in separate assays and determination of their combined amount or presence determines the amount or presence of antibodies to said immunogen.

Alternatively described, the first described step may be absent and said detection step may be performed on a blood sample obtained from an individual.

As used herein the terms “detecting” and “determining the amount or presence of” encompass both quantitative and qualitative assessment of the level of antibody production, in the sense of obtaining an absolute value for the amount of antibody produced in the sample, and also an index, ratio, percentage or similar indication of the level of antibody production, as well as semi-quantitative or qualitative assessments. The term “determining the presence of” encompasses also situations where a negative result, indicating the absence of antibodies, is of value in assessing the immune response of a subject.

The “combined” amount or presence refers to the addition of the results obtained, be they e.g. in numerical or qualitative form. Most preferably the antibodies are detected together and their combined amount or presence is the readout of the assay for target antibodies, which is assessed as a single measurement. As used herein “target” antibody refers to the antibody to which the assay is directed and which recognises a specific antigen (containing at least an epitope of the immunogen).

As referred to herein an “immunogen” is any entity which is present in the challenge to the immune system and is preferably a proteinaceous entity. Antibodies to said immunogen are those which specifically recognize an epitope on said immunogen or a part of said immunogen.

The “blood sample” as referred to herein is a sample of blood which may be obtained by any convenient means from any convenient source for analysis. Especially preferably the blood sample is a peripheral blood sample sourced from the vein of a limb, preferably an arm in the case of humans. Such a blood sample is not a whole blood sample, as discussed hereinafter and consists of blood from which at least one component, e.g. red blood cells have been removed.

“Plasma” as referred to herein corresponds to the fluid portion of blood and is substantially devoid of any cellular components. Plasma may be obtained by known techniques of preparation, e.g. by collection of the fluid portion of blood post-sedimentation or the supernatant after centrifugation of cellular components. Significantly, the plasma is substantially free of lymphocytes. As used herein, “substantially” refers to the absence of contaminating components to a high level, e.g. 90 to 95% free of contamination.

“Lymphocytes” as referred to herein in the context of the method concerns lymphocytes which produce antibodies, i.e. B lymphocytes. The method may however be performed in the presence of T lymphocytes and methods which refer to the preparation of isolated lymphocytes refer to the preparation of an isolated population of cells containing both B and T lymphocytes. Especially preferably however, the assay is performed on a substantially purified B lymphocyte population.

By ‘lymphocyte antibody’ it is meant antibody that is in the lymphocyte when the sample is taken, and/or that has not been secreted by the time the lymphocyte is assessed. Such antibodies will have been made in vivo in response to antigenic stimulation; and may continue to be made in the lymphocytes after removal of the sample, e.g. if they are incubated as in the method described in WO96/26443. It will be appreciated that during the time between removal of the sample and testing, some low level secretion may occur. At the time of analysis therefore those antibodies may or may not still be present in lymphocytes and may have been secreted during any in vitro procedures. The antibody which may be released from the lymphocytes by disruption is ‘newly synthesised’ antibody.

As used herein, the term “newly synthesised antibody” refers to an antigenically active antibody (i.e. capable of recognizing and binding to the antigen corresponding to the immunogen) which has been produced or synthesised by and within a lymphocyte cell in response to the presence of an immunogen in vivo as part of an on-going immune response. Thus, the antibody is synthesised by a lymphocyte during the course of an immune response triggered by the presentation of an immunogen in vivo, i.e. synthesised before and at the time the lymphocyte-containing sample is removed from the subject animal. This term is synonymous with ‘lymphocyte antibody’.

“Plasma antibody” refers to an antigenically active antibody (i.e. capable of recognizing and binding to the antigen corresponding to the immunogen) which has been produced or synthesised by and within a lymphocyte cell in response to the presence of an immunogen in vivo as part of an ongoing immune response and subsequently secreted into the blood from the lymphocytes of the individual, and are thus found only in the plasma of the sample. Thus, the antibody is synthesised by a lymphocyte during the course of an immune response triggered by the presentation of an immunogen in vivo, and is subsequently secreted into the blood. This secretion thus occurs before and at the time the sample is removed from the animal.

Plasma antibody is therefore distinct from the ‘lymphocyte antibody’ and newly synthesised antibody described above, in terms of where it is detected, although it is clear that once secreted, the ‘lymphocyte antibody’ will become plasma antibody, and the antibodies may be chemically or structurally identical. The definition simply reflects a snapshot of antibody distribution within the sample at the time it is taken.

Prior to secretion however, at least a portion of the population of lymphocyte antibodies may not have undergone post-translational modification and/or may not be in the mature form (see hereinafter) and these antibodies will thus be chemically and structurally different to those antibodies which are secreted and which are hence the plasma antibodies.

Reference herein to “antibodies or parts” thereof refers to antibodies or parts thereof which are antigenically active (i.e. capable of recognizing and binding to the immunogen or part thereof). In the case of lymphocyte antibodies this may be those entities which have not yet been secreted from the cells at the time of taking the sample and for example are not in the final mature form even though they already have antigenic capabilities. As mentioned previously, such lymphocyte antibodies and parts thereof will generally correspond to antibodies produced in the 2 hours preceding the assay and/or collection (which may occur exclusively in vivo or at least partially in vitro prior to cell disruption if the cells are kept under appropriate conditions), although the time course of the secretion pathway may vary.

Whilst antibodies may be produced rapidly within the cells (e.g. within 1-2 mins, although secretion is rather slower), free chains which make up antibodies or unglycosylated or partially glycosylated antibodies or antibody chains may be present in the lymphocytes, and hence released on their disruption. Where appropriate these “parts” may be detected and included in the measurement of the presence or amount of newly synthesised antibodies providing they are antigenically active as described above. Antibodies or parts thereof released from the lymphocytes comprise newly synthesised antibodies, or lymphocyte antibodies.

Plasma antibodies or parts thereof include those parts which retain antigenic activity (as described above), but which do not correspond to the secreted form, e.g. which have been partially degraded through proteolytic degradation.

As referred to herein, an “assay” is a suitable technique for the determination of the amount or presence of antibodies in a sample. Suitable assays for this purpose are described hereinafter.

The early, accurate and rapid detection of current and previous infection according to the invention is of particular use in a variety of applications, particularly applications in which this information is used to determine the utility of the sample or source organism. Thus, for example, the blood of animals may be examined to determine their suitability for export, import, travel, consumption, breeding etc., e.g. by examining for antibodies to challenge such as by Aphthovirus (the causative agent of foot-and-mouth disease).

The sample to be analysed may thus be from any animal from which blood may be derived. Preferably however said animal is a mammalian animal or a laboratory or agricultural animal. Preferably the animal is selected from the list comprising fish, chickens, ducks, geese, pheasant, rats, mice, rabbits, dogs, cats, goats, sheep, cows, deer, pigs, horses, donkeys and humans. Especially preferably the invention is directed to testing human samples.

Relatively small volumes of blood may be used for the assay. Lymphocyte antibodies can be detected in lymphocyte lysates from as little as 50 μl of blood. Conveniently therefore a sample of less than 10 mls of blood is required. Conveniently as little as less than 1 ml may be used for the method of the invention. Preferably however as little as 100-500 or 100-200 μl is employed.

Antigens or immunogens to which antibodies for detection according to the method of the invention are directed include both bacterial and viral antigens. Clinically important antigens include, but are not restricted to those from for example Herpes Simplex virus, Cytomegalovirus, human immunodeficiency virus (HIV) and any of the Hepatitis viruses as well as Toxoplasma (Toxoplasma gondii) and Epstein-Barr virus (EBV) tuberculosis, syphilis (Treponema pallidium) and chlamydia (Chlamydia trachomatis). In general, however, any immunogen arising as a result of challenge, such as by infection or vaccination, eliciting a clear antibody response may be detected by the method of the invention.

In cases where chronic infection results in detectable levels of target antibodies within lymphocytes and subsequent secretion of these antibodies into the plasma, assays for these antibodies may also be performed according to the method of the invention. Thus for example, antibodies to any immunogen which may be detected using a conventional ELISA method may be detected by the method of the present invention.

Detection of antibodies to such antigens could be used to rapidly establish whether patients are infected e.g. for blood screening purposes or for establishing and/or monitoring infection. Thus the invention further extends to the use of the method of the invention for diagnosing or monitoring infection of a human or non-human animal or a part of said animal by an immunogen, preferably a bacterium or virus, and the presence or extent of infection by said immunogen, preferably by said bacterium or virus, is determined by reference to appropriate control and/or reference samples.

Although the test is particularly suited to high-throughput analysis, the test may be used in any circumstance in which a yes:no answer is required, e.g. in a hospital, clinic, laboratory or a doctor's surgery. More commonly however, identical testing of multiple samples (e.g. of more than 10, e.g. more than 100 or 1000 samples) is contemplated, e.g. as would be performed for blood banks, especially in areas of the world with a high prevalence of blood-borne infectious diseases such as HIV and hepatitis, that can be passed onto recipients of the blood.

Blood banks require a rapid answer to whether the blood can be used for transfusion; there is no need to classify the infection into the particular stage or time post-infection. If it is found that a donor is infected, then their detailed diagnosis can be performed separately. Blood banks simply need to know whether the blood can be used or not. In a preferred aspect therefore, the method of the invention is applied to screening of multiple samples simultaneously or sequentially. Especially preferably said screening is automated, e.g. high-throughput screening is performed. In a particularly preferred feature, the method of determining the amount or presence of antibodies is used to determine the suitability of a sample for transplantation or inter-individual transfusion, particularly for example wherein the samples are aliquots of a blood stock obtained from one or more individuals, such as would be held by a blood bank.

It will be appreciated that the situation outlined for the detection of HIV infection is only one example of a wide variety of diseases, infections and immunisations, the detection of which will benefit from the application of this invention in detecting infection.

This assay offers several advantages and is of particular use in screening methods as it provides a rapid answer to the question of whether the individual has been or is currently infected. A positive answer to this question would given a positive diagnosis and for example exclude the donor or individual from blood donations, and subsequent analysis could then be performed to determine whether the infection is acute (i.e. antibodies are in the lymphocytes) or a past infection (i.e. antibodies are in the plasma).

The detection of lymphocyte antibodies may conveniently be performed by any means. For example, using the method of WO96/26443, active production and secretion of antibodies in vitro may be measured, following in vivo stimulation caused by exposure to infection or immunisation.

The detection of lymphocyte antibodies may alternatively and most conveniently be performed by disrupting the lymphocytes in said blood sample, e.g. as described in WO 00/77525, and as described hereinafter in more detail. A suitable method of preparing and analysing lymphocyte antibodies is disclosed in the Example of WO 00/77525.

Thus in a preferred aspect therefore, the invention provides a method of determining the amount or presence of antibodies to an immunogen in a blood sample comprising at least the steps of:

obtaining a blood sample (comprising plasma and lymphocytes);

disrupting the lymphocytes of said blood sample whereby to release antibodies or parts thereof associated with said lymphocytes; and

detecting the amount or presence of said lymphocyte antibodies or parts thereof released from the lymphocytes and the amount or presence of plasma antibodies;

wherein said plasma and lymphocyte antibodies are detected together or in separate assays and determination of their combined amount or presence determines the amount or presence of antibodies to said immunogen.

Alternatively described, the first described step may be absent and said detection step may be performed on a blood sample obtained from an individual.

The lymphocytes referred to herein are lymphocytes that are present in the whole blood sample which has been taken from the subject, and in the blood sample which is subjected to the method. The various manipulations and preparations that are carried out during the method may involve separating the lymphocytes, partially or wholly, from the other components of the blood sample. Thus, the lymphocyte disruption step need not always be carried out on the blood sample and may be carried out on a lymphocyte preparation derived from the blood sample. Reference to lymphocytes of said blood sample thus refers to lymphocytes which are or were present in the whole blood sample irrespective of whether they have been wholly or partially purified from the blood sample.

In a preferred embodiment, a single well assay is performed. In other words, the plasma antibody and lymphocyte antibody detection step is performed on a single sample. As mentioned above, this has the advantage of increasing antibody levels to detectable levels by the assay of both plasma and lymphocyte antibodies in a single sample when levels of one or both of these antibodies is at the detection threshold.

In this embodiment, the sample on which the detection step is carried out comprises both lysed lymphocytes and plasma. As a consequence, a single assay will determine whether there are any plasma antibodies or lymphocyte antibodies present in the sample. In view of the fact that these two antibody types will both bind to the detection antigen or antibody, no distinction is made in this assay between the two types of antibody.

It had not previously been appreciated that such a single well assay could be performed. The presence of both plasma and lymphocyte lysate might have been thought to have had a negative impact on the results that would be obtained, e.g. by dilution of antibodies which were present to levels below the threshold of detection. It has however been found by the inventors that it is possible to detect plasma antibodies and/or lymphocyte antibodies in a mixture of the two components without a significant loss of sensitivity.

The single sample, i.e. the blood sample, containing the lymphocytes and plasma antibodies may take a variety of forms ranging from a largely unpurified sample to substantially purified lymphocyte and plasma portions. As referred to herein such “portions” refer to those fractions of whole blood which contain the lymphocytes or plasma. These portions may contain in addition other components of the blood.

The detection step may thus be performed on a whole blood sample from which one or more of the components of the blood sample have been removed before the assay is performed to generate the “blood sample” used in the method of the invention. For example, in a preferred embodiment, red blood cells may be removed from whole blood samples by standard techniques, e.g. centrifugation or affinity-based removal.

Further components may also be removed, e.g. non-B lymphocytes. Conveniently such a sample may be obtained by positive selection of particular components, e.g. after gradient centrifugation the B lymphocyte-containing (e.g. buffy coat) and plasma-containing portions may be collected for assay.

As an alternative or in addition, the plasma and lymphocytes portions may be wholly or partially purified, together or separately, from the remainder of the whole blood sample using techniques known in the art. If they are purified from whole blood separately, when performing the single well assay they will be recombined i.e. mixed together prior to performing the single well assay.

In a preferred aspect therefore, starting with a whole blood sample isolated from an individual, a plasma-containing sample or portion is isolated and separately a lymphocyte-containing sample or portion is isolated. Such samples or portions may be generated starting from two aliquots of a whole blood sample or may be generated from a single aliquot in which that aliquot is separated into two discrete portions (e.g. a supernatant and cell pellet after sedimentation or centrifugation) from which the two isolations are subsequently performed. In the case of the single well assay the lymphocyte-containing and plasma-containing samples or portions that are generated are recombined for analysis.

In assays in which recombination of the plasma-containing and lymphocyte-containing samples or portions is performed, the aliquots for analysis are preferably recombined in a 1:1 volume ratio. However various combinations will be tolerated and conveniently any combination in the range of for example 1:0.4 to 1:4 for plasma:lymphocyte samples or portions may be used. Preferably, in view of the relative lower levels of lymphocyte antibodies, bias towards higher volumes of the lymphocyte sample or portions is preferred, e.g. a range of 1:1 to 1:4 plasma:lymphocyte sample or portion. The above ratios rely on the antibodies being present in the samples or portions used at the approximate concentration at which they would appear in whole blood (in which the lymphocytes had been lysed). As required, depending on the sensitivity of the assay used for detection and the amount of antibody present, sample or portion dilution may be performed. Thus for example in achieving a 1:1 ratio, for every 1 μl of blood, the plasma present in that 1 μl may be recombined with a lysate generated using the number of lymphocytes found in the original 1 μl blood sample. Thus, the amount of lymphocyte and plasma antibody present in a 1:1 ratio mixture from 1 μl of whole blood is approximately the same as the amount of antibody present in the 1 μl of whole blood. During the preparation of the samples for analysis, the dilution of the plasma-containing sample lymphocyte containing sample or lysate can be adjusted appropriately so that this ratio is maintained.

Lymphocytes may be separated using standard techniques known in the art, e.g. using filter methods or techniques using absorbent material or lymphocyte preparation kits. Thus, for example, various whole blood preparations may conveniently be used to obtain the lymphocytes, e.g. from heparinized blood, EDTA-blood etc., such as are routinely prepared in clinical laboratories.

Preferably the lymphocytes are separated using standard lymphocyte separation media e.g. Lymphoprep (Nycomed Pharma AS, Oslo, Norway), or using immunomagnetic separation (IMS) or a similar solid phase based separation system or other common techniques. In the case of IMS or similar separation techniques, a solid phase e.g. magnetic beads coated with antibodies specific for certain sets of leucocytes may be used to separate the useful lymphocytes selectively. Preferably anti-CD19 antibodies are used for B-cell isolation. Where separated lymphocytes are used, the cells may be washed prior to use, using standard washing methods.

Plasma may be prepared by any convenient means, such as by centrifugation (e.g. 1000×g for 5 minutes) of whole blood samples or simply allowing the whole blood to stand and the cellular components to form a sediment.

Plasma- and lymphocyte-containing samples may be enriched for that component or may be substantially purified. As referred to herein “substantially purified” samples refer to those in which other components are present to a relatively minor extent, e.g. make up <20%, preferably <10 or <5% of the total volume or weight of the sample. Thus, for example, some contamination of plasma by cellular debris, may be expected and tolerated, possibly including lymphocytes and their contents. Similarly lymphocyte preparations may contain some other cells and/or plasma components. These should however preferably be minimized, e.g. <1%.

For the single well assay, when separate plasma and lymphocyte portions are generated for recombination, the wholly or partially purified lymphocytes may be disrupted before or after addition to the plasma portion from the sample.

After the analysis of the combined antibodies, if a sample yields a positive result, further information regarding the onset or stage of the immune response may be generated by re-testing separate lymphocyte and plasma fractions.

As an alternative to the single well assay, the assay of lymphocyte and plasma antibodies may be performed separately in two assays and the information obtained in those assays may be combined. Preferably such assays are conducted simultaneously. In this case, the plasma and lymphocyte samples may be prepared as described hereinbefore for the single well assay, and simply tested separately by any means as described herein without recombination. In such cases the plasma and lymphocyte samples are substantially absent contaminating lymphocyte or plasma antibodies, respectively (e.g. <20%, preferably <10 or 5% relative to the amount of the other antibody form). Preferably however the assay is conducted using the single well format.

By “disrupting” the lymphocytes is meant that the cell contents including any synthesised antibodies are released from within the confines of the cell membrane and internal membrane structures such that they may be detected by any convenient biochemical or chemical assay. It is known that immunoglobulins are synthesised and secreted during a pathway through the endoplasmic reticulum and Golgi complex. Thus, necessarily disruption requires release from these internal structures. Thus disruption of the lymphocytes may be achieved by known methods of cell disruption which effectively disrupts external and internal membrane structures without affecting the ability of the released antibodies to bind to their complementary epitopes, e.g. by using physical disruption means or cell-disrupting buffers or solutions.

Disruption may be achieved by the use of chemical means using for example detergents, chaotropic agents, disruption buffers e.g. containing EDTA or alternative disruption methods such as sonication or physical disruption through generation of shear stresses.

Preferably, however a disruption buffer is used as this is generally the simplest and most convenient technique e.g. as described in the Examples herein i.e. buffer containing detergent such as Tween20 and/or NP-40. Preferably Tween is used at a final concentration of 0.01 to 0.1%, especially preferably at around 0.05% and/or NP-40 is used at a final concentration of 0.1 to 1%, especially preferably at around 0.5%. Appropriate disruption buffers may be used to stabilize the released antibodies, e.g. to control pH or degradation. Thus for example buffers containing protease inhibitors may be employed if necessary.

Incubation with the disruption buffer is performed at an appropriate time to maximize disruption, e.g. at room temperature for 3 to 15 minutes, e.g. for around 5 minutes.

Alternative methods of disruption include for example the use of freeze/thaw cycles or even the use of liquid nitrogen. This results in a lysate in which the released antibodies are in solution which is used for subsequent steps.

It will be appreciated that in order to obtain sufficient amounts of newly synthesised antibodies in the sample to be detected, it is desirable that as many of the lymphocyte cells as possible are disrupted to release the antibodies. Preferably, then, the disruption means is suited to this end and at least 40% or 50%, more preferably at least 60%, 70% or 80% and more preferably at least 90% of 95% of the lymphocytes in the sample are disrupted. After disruption of the lymphocytes the antibody content of the sample is assessed by an appropriate technique allowing detection of the target antibodies as described hereinafter.

Detection of the lymphocyte and plasma antibodies may be by any method which allows for identification of those antibodies which bind to the immunogen of interest (or an epitope thereof) causing the immune response. Thus any detection technique which results in the production of a signal which reflects the presence of the target antibody may be used. For example enzyme-linked assays may be used in which a soluble or insoluble product may be produced from a substrate, whose amount may be assessed.

Conveniently the lymphocyte and plasma antibodies may be detected for example by means of a solid phase binding assay, e.g. an ELISA, wherein they bind to the antigen used in the assay, although the antigen used may be different to the immunogen stimulating the immune response in the first place. Thus, whilst both the antigen used in the assay and the immunogen which has stimulated or is stimulating the production of antibodies in vivo would bind to the antibodies to be detected by virtue of identical or very similar epitopes, in other respects the antigen and immunogen may not be identical. Thus, the antigen used in the method of the invention may be material containing all or some parts of the relevant immunogen, e.g. derived from infected individuals, or purified parts from the same or similar material and may be prepared synthetically, e.g. by chemical synthesis or recombinant expression, with added or deleted portions relative to the native antigen. Thus fusion proteins, or molecules expressing only the appropriate epitope(s) may be used.

Conveniently, for the detection step, the sample may be contacted with a solid phase carrying an appropriate binding partner to immobilize the antibody or antibodies to be detected. As referred to herein a “binding partner” is one of two partners which together form a binding pair and which bind selectively and specifically to one another, e.g. a ligand and receptor or an antigen and antibody. Conveniently the binding partner is the antigen (immunogen or part thereof) or antigens (i.e. one or more), recognised by the antibody or antibodies or parts thereof to be detected.

In a further alternative however, different binding partners which are not the target antibodies' antigens may be used, for example protein A, protein G or antibodies which recognise and bind to the antibody to be detected. In the latter case, highly specific binding is not required as specificity may be introduced in this embodiment of the assay method by the subsequent binding of antigens, which bind specifically to the antibodies to be detected, during the detection step. Thus, in all embodiments a specific antigen-antibody complex is created. The presence of such complexes, preferably immobilized to a solid support is ascertained in the detection step of the methods of the invention.

Thus in a preferred feature the detection step of the method of the invention comprises detection of the antibodies or parts thereof by the formation of an antibody:antigen complex wherein said antigen (which preferably is not an antibody) comprises or contains the immunogen or a part thereof containing at least the epitope of the immunogen.

In a particularly preferred aspect, the step of detecting the amount or presence of plasma or lymphocyte antibodies or parts thereof comprises contacting a plasma-containing portion of said sample and a lymphocyte-containing portion of said sample with one or more antigens, preferably carried on a solid phase, wherein the antigens are recognised by the antibody or antibodies to be detected. Alternatively one or more antibodies which recognise the target antibody or antibodies to be detected may be contacted with said portions. As described herein said portions of the sample may be present in a single sample and their analysis may be carried out simultaneously by binding to the same solid support. Alternatively, said portions may be discrete and separate and the antibodies may be detected in those portions in separate assays.

In this and other methods described herein, the amount of antibody binding may be determined by comparison to control and/or reference samples. Appropriate control or reference samples may be negative or positive controls, e.g. blanks, normal samples or spiked samples.

The solid phase, when employed, may be any of the well-known supports or matrices which are currently widely used or proposed for immobilisation, separation etc. These may take the form of particles, sheets, gels, filters, membranes, or microtitre strips, tubes or plates etc. and conveniently may be made of a polymeric material. However, for ease of operation and simplicity standard microtitre plates and wells may conveniently be used, preferably standard ELISA plates.

The solid phase may also be modified to permit detection of antibodies specific for a range of different antigens. Thus for example, discs or strips etc. of a suitable solid phase material e.g. nitrocellulose or such like may be coated with different antigens and added simultaneously to a microtitre well or other suitable vessel, not containing any contacting antigen. Antibody binding detection methods may then be used to distinguish between the different antigens. Thus the invention extends to the use of multiple solid phases each bearing a different antigen or antibody which is recognized by, or recognises, a different target antibody.

Conveniently when sandwich-type assays are employed, the solid phase carries one or more antigens (solid phase antigens) recognized by the antibody or antibodies or parts thereof to be detected (target antibodies). Alternatively, the solid phase may carry one or more antibodies (solid phase antibodies) which recognise the antibody or antibodies or parts thereof to be detected (target antibodies). To allow detection according to the method of the invention, as appropriate, depending on whether the said solid support carries antibodies or antigens as described above, one or more antigens, recognised by the target antibodies immobilised on said solid phase, are contacted with said solid phase or alternatively one or more antibodies, which recognise target antibodies immobilised on said solid phase, are contacted with said solid phase. These antigens or antibodies which then become bound to the solid support may be appropriately labelled to allow detection as described hereinafter.

Sets of discs each coated with relevant antigens consistent with a certain clinical condition or syndrome may be used in order to identify which of the suspected agents is causing the disease. The discs would then be individually processed in separate wells. This is a particularly material-saving procedure, since tests can be performed for simultaneous testing of a multiplicity of different antigens (either from the same infectious agent or from different agents relevant for the clinical syndrome or condition in each case) using the same small blood volume. An alternative approach is to use multiple samples e.g. comprising disrupted lymphocytes and plasma in multiple wells, each coated with different binding partners, e.g. antigens or antibodies, and develop the test accordingly.

Techniques for binding of the binding partner, e.g. antigen to the solid phase are also extremely well known and widely described in the literature. Many standard antigen coating procedures are described for example in ELISA and other solid phase Immunoassays, Theoretical and Practical Aspects; 1988, ed. D. M. Kemeny & S. J. Challacombe, John Wiley & Sons. If desired, the plates may be washed and blocked, again using standard techniques. Thus, for example, standard microtitre plates e.g. ELISA plates may simply be coated with binding partner by incubating the plates overnight at 4° C. in a suitable buffer e.g. phosphate buffered saline (PBS) containing the binding partner e.g. at concentrations of 0.01 to 150 μg/ml protein, followed by blocking using appropriate blocking media (generally a neutral buffer, such as PBS, containing a blocking protein e.g. calf serum or proteins from dried milk) and incubating e.g. at 37° C. for 1 to 5 hours. After removing the blocking solution the plates are ready for use.

Conveniently, the materials and means required to perform the method of the invention may be provided in kit form, e.g. containing a binding partner-coated solid phase for the detection step, a disruption buffer and a solid phase carrying an appropriate binding partner for lymphocyte isolation. Where the solid support is supplied it may be ready coated with binding partner and appropriately blocked.

The binding of the antibody to its antigen is then detected. The detection step, in terms of reading the signal, conveniently takes place in solution. However, an insoluble product or signal may be generated which is not read in solution. Any of the known means of detecting antibody binding may be used, as long as a readable signal is generated; for example depending on fluorescence, chemiluminescence, colorimetry or an enzyme reaction to produce the detectable signal. Where a solid phase is not used, the target antibodies may be detected by any other sensitive serological method such as light scattering (e.g. nephelometry) and resonance procedures. Conveniently, an immunoassay may be used as the means of detection, and preferably an enzyme-linked immunosorbent assay (ELISA), such as the Abbott PRISM ELISA (Abbott Laboratories, Chicago, USA) or the Ortho ELISA (Ortho Clinical Diagnostics, New Jersey, USA). However, test procedures other than ELISA are contemplated within the scope of the invention for detecting antibodies. Techniques which use coated discs or glass plates, for example, flooded with a suspension of disrupted lymphocytes and/or plasma may be suitable. Any standard technique for detecting antibodies known in the art, such as techniques which result in an insoluble or soluble product, may be adopted for use in the method of the invention either for quantitative analysis or for a qualitative (e.g. yes/no) type of test.

Immunoassay, and particularly ELISA, techniques are well known in the art and described in the literature (see for example ELISA and other solid phase Immunoassays, Theoretical and Practical Aspects; 1988, ed. D. M. Kemeny & S. J. Challacombe, John Wiley & Sons).

Following the contacting of the disrupted lymphocyte sample, an enzyme-antibody conjugate may be added, for example in the ELISA detection method, which binds to the antibody bound to the antigen on the solid phase. Similarly, if the antibody to be detected is bound to the solid phase non-specifically via a binding partner, for example by an antibody against antibodies of a different species, an enzyme-antigen conjugate may be added which will bind specifically to the immobilized antibody to be detected. An enzyme substrate is then added in order to develop the detectable signal. In the present invention, a soluble substrate is conveniently used, yielding a signal detectable in solution. This is advantageous since it facilitates and simplifies the handling and processing of a large number of samples, and permits estimation of antibody production, although as mentioned above, absolute quantitation is not necessary, and if desired a qualitative or semi-quantitative result may be obtained.

For convenience the substrate may be selected to yield a spectrophotometrically detectable signal, which may simply be read by reading absorbance, e.g. using a standard ELISA plate reader. Indeed, standard ELISA reagents may be used, which has the advantage of rendering the assay of the invention compatible with existing methods and techniques routinely employed in clinical laboratories. However, other detection/signal generating systems may be used, yielding signals detectable by fluorescence, chemiluminescence etc.

Immuno-enzymatic amplification methods may also be used to improve the signal and increase sensitivity, for example using avidin-biotin methods such as the Extravidin system available from Sigma. Biotinylated secondary antibodies are used as ELISA reagents, in combination with a peroxidase avidin complex. Since one molecule of avidin is capable of binding several molecules of biotin, the use of avidin-biotin peroxidase complexes increases the surface concentration of peroxidase molecules, giving the method even greater sensitivity.

In order to ensure that the assay method of the invention is working confidently, appropriate controls may be included and used for determining the presence or amount of antibody. For example to ascertain that the recorded signal is not due to sporadically and nonspecifically (bystander) activated lymphocytes, a negative control antigen may be used. This antigen would be from an infectious agent most unlikely to be responsible for the disease or infection under investigation, e.g. tetanus toxoid. The number of such bystander activated lymphocytes should in any event be lower than required for a positive result.

One benefit of the invention in which lymphocyte antibodies are detected following lymphocyte disruption has been the avoidance of any incubating step or special assay conditions. Thus, the lymphocytes in said sample are preferably not incubated under conditions to allow production and/or secretion of antibodies prior to the method of the invention, i.e. after collection in the whole blood sample or derived preparation. Conditions which allow production and/or secretion are for example incubation at >4° C., e.g. from 20 to 39° C., e.g. around 37° C. for >5 seconds, e.g. 30 seconds to 10 minutes, or longer. The absence of an incubation step means that samples may conveniently be treated after sampling in order to effect disruption or lysis of the cells, and the lysate can then be stored e.g. by freezing or refrigerating for a period of time before the assay step is carried out.

Alternatively, as mentioned previously, the sample e.g. whole blood from which a component has been removed, a semi-purified (e.g. buffy coat) or a purified preparation can be stored e.g. by freezing or refrigeration (as appropriate, depending on the sample) for a period of time e.g. for up to several hours or days, e.g. for more than 4 hours, e.g. from 6 hours to 1, 2, 3 or 4 days before the lymphocytes are disrupted. For example, a purified lymphocyte preparation could be stored by freezing for a period of time e.g. for several hours or days, or by refrigeration, e.g. for a few hours, if necessary or desired, before the sample is treated to disrupt the cells. Thus for example, purified lymphocytes could be stored at >0° C.≦4° C. (e.g. at 4° C.) for several hours e.g. for up to 4 to 6 hours before treating them to disrupt or lyse the cells. Plasma may be similarly stored.

However, it has been found that some samples e.g. whole blood preparations can be stored under refrigeration for longer periods of time without adversely affecting the cells and interfering in the test results. For example it has been found that whole blood samples can be stored under refrigeration (e.g. at 4° C.) for at least 6 days, and if desired the samples can be intermittently kept at room temperature (18-25° C.) for at least 6 hours, before procedures for purifying lymphocytes are initiated, without adversely affecting the performance of the test to detect antibody.

This is particularly useful when blood samples are on hold in the laboratory and are stored under refrigeration while results from routine or supplementary plasma/serum testing procedures are pending. This implies that the physical integrity of the lymphocytes is maintained during storage, thus allowing subsequent lymphocyte purification from the stored blood (if desired) and disruption of the lymphocytes to take place, without a significant reduction in the antibodies being detected.

If samples are to be taken and stored for long periods then the standard approach is to separate the lymphocytes, disrupt them, and store the lymphocyte lysate and plasma separately by freezing.

A new alternative to this is to freeze whole blood. This can be performed by adding a solution to preserve lymphocyte integrity during freezing, e.g. a solution containing DMSO. The Examples describe the addition of an equal volume of buffer containing citrate+20% DMSO to the whole blood prior to freezing. The DMSO protects the cells from lysing during the freezing procedure and, when thawed, they can be treated as normal samples. Thus in a preferred aspect, collected samples are frozen in a solution containing a final concentration of from 5-15% DMSO, preferably 7.5-12.5%, e.g. 10%. Optionally the DMSO may be supplemented with polyethylene glycol, in which case the DMSO concentration may be lowered, e.g. to 3-7%, e.g. 5%.

Thus, in a preferred embodiment prior to disruption of the lymphocytes, e.g. the blood sample may be stored for several days e.g. up to about 6 days or more, especially when stored under refrigeration at about 4° C. (e.g. from 1 to 6° C.) or less even allowing for removal of the sample from its refrigerated environment for intermittent periods of e.g. 4 to 6 hours for example at room temperature on two or more occasions during the storage period, without adversely affecting the results of the assay. This is particularly useful when blood samples are stored under refrigeration but where it is unavoidable that the sample be left on the bench at room temperature for a short time. Surprisingly, it has been found that cell viability is not sufficiently affected to interfere with or prevent acceptable results being obtained according to the assay method of the invention. In a further preferred embodiment, when purified lymphocyte preparations are used, these samples (and/or plasma) may be stored at less than 4° C. (e.g. >0 to 4° C., e.g. for several days or longer) or stored at above 4° C. (for up to 6 hours).

The possibility of storing various samples for use in the assay makes the method of the invention particularly suitable for larger diagnostic laboratories when expensive automated laboratory equipment e.g. ELISA hardware can be most efficiently run using a substantial number of samples at one time. It also means that samples may be taken in many different locations and mailed or delivered to a central diagnostic laboratory for analysis in a similar manner to procedures set up for analysis of chemical serum samples in other types of tests.

In preferred aspects of the invention, a single well assay is used in which the whole blood sample is divided into two portions from which a plasma sample and a lymphocyte sample is prepared or lymphocyte- and plasma-containing portion are separated from a single whole blood aliquot. The lymphocytes are then disrupted and the samples recombined and the levels of antibody assessed.

Thus in a particularly preferred aspect, the present invention provides a method of determining the amount or presence of antibodies to an immunogen in a blood sample comprising at least the steps of:

obtaining a whole blood sample;

isolating plasma from an aliquot of said sample;

isolating lymphocytes from an aliquot of said sample;

combining said plasma and lymphocytes;

disrupting said lymphocytes to release antibodies or parts thereof associated with said lymphocytes (which step may optionally be performed prior to combination);

and detecting the amount or presence of said lymphocyte antibodies or parts thereof released from the lymphocytes and the amount or presence of plasma antibodies in a single assay;

wherein the combined amount or presence of said plasma and lymphocyte antibodies determines the amount or presence of antibodies to said immunogen.

Especially preferably said lymphocytes are isolated by affinity binding to a solid support (such as beads, e.g. magnetic beads) via a binding partner on said solid support which binds specifically to its binding partner on said lymphocytes, e.g. said solid support carries anti-CD19 which bind to the complementary molecule on the lymphocytes. Especially preferably, disruption is performed using a detergent, particularly preferably NP-40 and/or Tween 20. Especially preferably detection of said target antibodies is achieved by contacting the target antibodies with one or more antigens on a solid phase. Particularly preferably said whole blood sample and/or said plasma or lymphocyte isolated therefrom are frozen prior to the assay. Further preferably, the method of the invention is conducted on multiple samples, e.g. from multiple individuals.

The invention will now be described in more detail with reference to the following non-limiting Examples in which FIG. 1 shows detectable events in HIV infection.

### EXAMPLE 1

Manual Method

Preparations

Preparation of Citrate Stock Solution (10× Citrate)

7 g Trisodium Citrate Dihydrate and 2.5 g. Citric Acid is dissolved in 90 ml distilled water. The final volume is brought to 100 ml. The solution is stored at 2-5° C.

Preparation of PlasmAcute Buffer

Phosphate Buffered Saline (PBS) pH 7.2±0.5. 100 μl of Tween20 is added per liter of PBS and mixed well. 10 ml of Citrate stock solution is added per 90 ml of the mixture above. This should be kept cool when not in use.

Preparation of PlasmAcute Buffer with Milk-Powder

10 g of fat-free milk powder (Clover Elite Fat Free) is added per 100 ml of PlasmAcute buffer and mixed well. The foam is allowed to settle before use. “Fat free” milk is in general less than 1% fat. This reagent should be made fresh before each run and kept cool when not in use.

Preparation of 100 ml 10× Disruption Buffer

| Stock solutions | 100 ml 10× buffer | |
|---|---|---|
| 5M NaCl | 30 | ml |
| 1M Tris | 10 | ml |
| 100% NP-40 | 5 | ml |
| Tween 20 | 0.5 | ml |
| Distilled water | 54.5 | ml |

Preparation of Magnetic Beads

The number patient samples to be processed (EDTA blood) is counted. 1 ml PlasmAcute buffer is added to an Eppendorf tube. The necessary volume of magnetic beads (One-Lambda Fluorobeads carrying covalently bound mouse monoclonal anti-CD19 antibodies) is calculated: 10 μl per sample+5 μl extra. The calculated volume (10 μl per sample+5 μl extra) of magnetic beads stock solution is added to the Eppendorf tube.

The solutions are mixed gently and the tube is placed on the magnetic stand. The fluid is gently removed from the tube while it is sitting in the stand. The tube is removed from the stand. The same volume of PlasmAcute buffer as the volume of stock solution added initially is added.

Separation of Lymphocytes

Eppendorf tubes corresponding to patient samples being processed are labelled. 635 μl of PlasmAcute buffer is added to the tubes. 10 μl of the magnetic bead suspension prepared earlier is added. 350 μl of non-clotted blood is added to the corresponding labelled tube and mixed gently.

The tubes are rotated gently once per second by turning the tube end-over-end for 5 minutes. The tubes are placed in the magnetic stand for 5 minutes (not more).

The fluid (WASH1) is gently aspirated from the tubes while sitting in the stand and discarded. The tubes are removed from the magnetic stand.

1,400 μl of PlasmAcute buffer is added and the washing step is repeated a further 3 times with 3 minutes in the magnetic stand. The final beads are suspended in 175 μl of PlasmAcute buffer.

Cell Lysis

18 μl of 10× disruption buffer is added to the 175 μl of cell suspension. This is mixed thoroughly with a pipette by sucking up and down and left at room temperature for 5 minutes. 175 μl of PlasmAcute buffer with 10% milk powder is added.

Preparation of Plasma

Plasma is prepared from the whole blood sample by centrifugation (1000 g, 5 minutes) and the plasma recovered.

Antibody Detection

This is performed on the lysate and plasma, mixed together or in parallel.

400 μl sample is applied to the Abbott PRISM ELISA system (Abbott Laboratories, Chicago, USA). Alternatively, MUREX's microtitre HIV ELISA—HIV ½ Group 0 may be used for analysis.

### EXAMPLE 2

Automated Method

The method of the invention is conveniently adapted for automation and the following method describes such a protocol using BioRobot M48 to prepare the lymphocyte lysate for analysis.

General Outline

Before running the protocol, the user must place blood samples (400 μl of each sample to be treated), plasticware and reagents (in plastic reagent containers) in the appropriate positions on the robot. When started, the robot will dilute the samples with an equal volume of the isotonic Buffer 1. The robot will then add CD19 beads to the diluted samples and incubate with mixing for 5 minutes to allow the cells to bind to the beads. Magnetic separation is performed, retaining the bead/cell complexes and discarding the liquid. The bead/cell complexes are washed twice in Buffer 1 and finally resuspended in Buffer 2, which will disrupt the cell membrane and release intracellular antibodies. Buffer 2 leaves the nucleus intact, thus avoiding the increase in viscosity that would otherwise be a technical challenge. The final stage of the protocol is a dilution of lysate with Buffer 3, performed to enhance storage stability and compatibility with downstream applications.

Definitions

Block: BioRobot M48 has a six channel pipetting head, and consequently works with six samples simultaneously. All worksurface positions that are directly associated with such a set of six samples is called a block. A block consists of a disposable assay tray with seven wells for each sample, six dedicated sample positions (these are not used in the PlasmAcute protocol, since they are at ambient temperature), six positions (a row) on the heating block and six positions (a row) on the elution block (temperature controlled).

Reagent containers: Plastic trays in which the user fills reagents for the run. The BioRobot M48 has seven small and four large reagent containers.

Buffer 1: Preferably PBS/Citrate/Tween

Buffer 2: Preferably 1× disruption buffer

Buffer 3: Preferably PBS/Citrate/Tween/Milk Powder

Magnetic separation: In general terms this a procedure in which paramagnetic beads and any substance associated with them are attracted to a magnet, and the liquid phase in which the beads were originally suspended is removed. On the BioRobot M48 this is achieved by aspirating the mixture of liquid phase, beads and cells and then applying a magnet to the pipetting tip to retain beads and CD19+ cells while dispensing everything else.

Washing step: Resuspending the beads in washing buffer and then performing a magnetic separation.

prologue-file: This is a part of the BioRobot M48 protocol where each step is performed for all selected blocks before going on to the next step. The prologue-file is commonly used to dispense reagents to assay trays and other block positions before the treatment of the samples start.

Block-file: This is a part of the BioRobot M48 protocol where all steps are performed for one block before going on to the next selected block. The block-file is commonly used for the treatment of the samples.

Epilogue-file: This is a part of the BioRobot M48 protocol where each step is performed for all selected blocks before going on to the next step. The epilogue-file is commonly used to perform operations after the sample treatment proper, like dilution resetting of heat- and elution block temperatures and returning stepper motors to home position.

Description of the Protocol

Prologue-File

P2: Beads in small reagent container 1 are resuspended to counter any settling and ensure uniform distribution of beads

P2: Beads are aspirated from small reagent container 1

P2: 100 μl beads are dispensed in well 1 of all assay trays that are to be used

P3: 900 μl Buffer 1 is transferred from large reagent container 1 to each of well 2 and 3 of all assay trays that are to be used

P4: 400 μl Buffer 1 is transferred from large reagent container 2 to each tube of all rows that are to be used on the heating block

P5: 180 μl Buffer 2 is transferred from small reagent container 2 to each tube of all rows that are to be used on the elution block

Block-File

B2: Beads in well 0 are resuspended and transferred to heating block

B3: Blood/Buffer 1/beads are incubated in the heating block with mixing for 5 min

B4: Blood/Buffer 1/beads are aspirated, the beads are magnetically retained in the tip, and the liquid is discarded

B5: Beads/cells are resuspended in Buffer 1 in well 2

B5: Buffer 1/beads/cells are aspirated, the beads are magnetically retained in the tip, and the liquid is discarded

B6: Beads/cells are resuspended in Buffer 1 in well 3

B6: Buffer 1/beads/cells are aspirated, the beads are magnetically retained in the tip, and the liquid is discarded

B7: Beads/cells are resuspended in 180 μl Buffer 2 in tubes on the elution block; cell membranes are lysed

Epilogue-File

E2: 520 μl Buffer 3 is transferred from small reagent container 3 to each tube of all rows that have been used on the elution block

E2: Temperature of elution block is reduced to 8° C. for storage.

### EXAMPLE 3

Freezing of Whole Blood Before Processing

The effect of freezing whole blood cells was determined. The freezing mixture used was PlasmAcute buffer with citrate (see Example 1) with or without 20% DMSO

An equal volume of freezing mixture was added to whole blood. The mixture was then frozen and stored (at either −20° C. or −70° C. The sample was then thawed to 2-8° C. and treated as fresh whole blood (NB 50% cells/unit volume).

Four replicates of whole blood per storage temperature were stored overnight at the appropriate temperature and then an equal volume of all samples were processed to recover B cells by standard methodology (see Example 1). Recovered B-cells were counted microscopically using a haemocytometer. Results are expressed as the mean of 4 replicates.

| | | Mean of Recovered Cells | Mean Cells/ml recovered |
|---|---|---|---|
| Storage | | [per area under | [corrected for DMSO |
| temp | DMSO | microscope] | dilution] |
| 4° C. | No | 25 | 25,000 |
| 25° C. | No | 56 | 56,000 |
| −20° C. | Yes | 20 | 40,000 |
| −70° C. | yes | 18.25 | 36,500 |

Thus there is no significant difference in processing a fresh sample or one frozen in the presence of DMSO to recover B-cells.

### EXAMPLE 4

Mixing of B-Cell Lysate and Plasma

The effect of recombining lymphocyte lysate and plasma on the sensitivity of the assay was analysed. All samples were analysed on the Abbott prism system. The data shown is the result of two replicates and 6 samples. Mean values are given. Negative plasma and lymphocyte lysates were prepared as described in Example 1 and recombined, or combined with buffer as set out below to test the effect of the recombination on background levels.

| | |
|---|---|
| 300 ul Plasma + 100 ul 2× disruption buffer | 0.34 sig/cov |
| 300 ul Plasma + 100 ul disruption buffer | 0.35 sig/cov |
| 300 ul Plasma + 100 ul homologous lysate | 0.4 sig/cov |
| 400 ul Plasma alone | 0.38 sig/cov |

Sig/cov is the signal produced in the ELISA, divided by the cut off value, such that any value of sig/cov of greater than 1 is treated as a positive result and any value of sig/cov of less than 1 is a negative result. The cut-off value is statistically determined for each experiment, based on the signals produced by analysis of negative control samples.

There was no change in value of a negative plasma sample due to the presence either of lysate or of the disruption buffer.

Sample recombination was also tested on a positive sample. The antibody levels in plasma and lymphocyte lysate from a single patient were first determined separately, and then after recombination at various ratios.

HIV positive sample plasma=24.5 sig/cov

B-cell lysate from same patient=0.42 sig/cov

Two replicates were tested and the mean values are given below.

Positive plasma + homologous lysate

| | | | |
|---|---|---|---|
| 400 ul | 400 ul = | 10 | sig/cov |
| 400 ul | 0 = | 24.5 | sig/cov |
| 300 ul | 100 ul = | 19 | sig/cov |
| 200 ul | 200 ul = | 11 | sig/cov |
| 100 ul | 300 ul = | 7 | sig/cov |
| 0 | 400 ul = | 0.42 | sig/cov |

It can be seen that the only reduction in signal is due to the dilution effect of the addition of buffer to the positive sample i.e. there is no inhibition of the signal produced by the assay by recombining lysate and plasma.

Practical variations of the above, which are appropriate for use on the Abbott prism system are as follows:

400 ul plasma+400 ul lysate; mix;→400 ul minimum into PRISM ELISA

250 ul plasma+250 ul lysate; mix;→400 ul minimum into PRISM ELISA
NB Only 100 ul is tested in the assay

1-39. (canceled)

40. A method of determining the amount or presence of antibodies to an immunogen in a blood sample comprising the steps of:
(i) detecting the amount or presence of plasma antibodies or parts thereof and lymphocyte antibodies or parts thereof in said blood sample, wherein said plasma and lymphocyte antibodies are detected together or in separate assays; and
(ii) determining the combined amount or presence of said plasma and lymphocyte antibodies or parts thereof, which determines the amount or presence of antibodies to said immunogen.

41. The method of claim 40, further comprising disrupting the lymphocytes of said blood sample to release antibodies or parts thereof associated with said lymphocytes.

42. The method of claim 40, further comprising the steps of:
(i) isolating a plasma containing sample from said blood sample; and
(ii) isolating a lymphocyte containing sample from said blood sample.

43. The method of claim 41, further comprising the steps of:
(i) isolating a plasma containing sample from said blood sample; and
(ii) isolating a lymphocyte containing sample from said blood sample.

44. The method of claim 40, wherein said blood sample is a whole blood sample, which is divided into two portions for the preparation of a lymphocyte containing sample and a plasma containing sample.

45. The method of claim 40, wherein a single whole blood aliquot is used to prepare a lymphocyte containing sample and a plasma containing sample.

46. The method of claim 42 or 43, wherein said lymphocyte containing sample and/or said plasma containing sample is a purified preparation.

47. The method of claim 43, wherein said plasma containing sample and/or said lymphocyte containing sample are recombined prior to disrupting the lymphocytes.

48. The method of claim 43, wherein said plasma containing sample and/or said lymphocyte containing sample are recombined after disrupting the lymphocytes and prior to said detection step.

49. The method of claim 47, wherein the ratio of said plasma containing sample and lymphocyte containing sample is from about 1:0.4 to about 1:4.

50. The method of claim 49, wherein the ratio of said plasma containing sample and lymphocyte containing sample is 1:1.

51. The method of claim 40, wherein said plasma and lymphocyte antibodies are detected in a single assay.

52. The method of claim 40, wherein said plasma and lymphocyte antibodies are detected in separate assays.

53. The method of claim 40, wherein said blood sample is a mammalian blood sample.

54. The method of claim 53, wherein said blood sample is a human blood sample.

55. The method of claim 40, wherein said blood sample is less than 1 ml.

56. The method of claim 40, wherein red blood cells are removed from said blood sample.

57. The method of claim 40, wherein non-B lymphocytes are removed from said blood sample.

58. The method of claim 40, wherein said immunogen results from infection or vaccination.

59. The method of claim 58, wherein said immunogen is a bacterial or viral antigen.

60. The method of claim 59, wherein said antigen is selected from the group consisting of antigens from Herpes Simplex Virus, Cytomegalovirus, human immunodeficiency virus (HIV), a hepatitis virus, Epstein-Barr virus, Aphthovirus, Toxoplasma, tuberculosis, syphilis, and chlamydia.

61. The method of claim 41, wherein the lymphocytes are disrupted by using chemical disruption buffers or physical disruption means.

62. The method of claim 61, wherein the chemical disruption buffers contain detergent.

63. The method of claim 40, wherein said antibodies or parts thereof are detected by contacting said antibodies or parts thereof with one or more antigens or antibodies, which recognize said antibodies or parts thereof.

64. The method of claim 63, wherein said antigens or antibodies, which recognize the antibodies or parts thereof to be detected, are carried on a solid phase.

65. The method of claim 40, wherein the released antibodies are detected by means of a solid phase binding assay.

66. The method of claim 64, wherein the solid phase of said solid phase binding assay carries one or more antigens recognized by the antibodies or parts thereof to be detected.

67. The method of claim 64, wherein the solid phase of said solid phase binding assay carries one or more antibodies recognized by the antibodies or parts thereof to be detected.

68. The method of claim 40, wherein the detection step is performed by immunoassay.

69. The method of claim 68, wherein the immunoassay is Enzyme-Linked Immunosorbent Assay.

70. The method of claim 67, wherein one or more antigens recognized by the antibodies to be detected are contacted with said solid phase and wherein said antibodies to be detected are immobilized on said solid phase.

71. The method of claim 66 or 67, wherein one or more antibodies recognized by the antibodies to be detected are contacted with said solid phase and wherein said antibodies to be detected are immobilized on said solid phase.

72. The method of claim 40, wherein said detection step takes place in solution.

73. The method of claim 40, wherein a soluble substrate is used for the detection step and yields a spectrophotometrically detectable signal.

74. The method of claim 40, wherein a negative control is used.

75. The method of claim 64, wherein multiple solid phases are employed, each bearing a different antigen or antibody, which recognizes a different antibody to be detected.

76. The method of claim 41, wherein the blood sample is stored at a temperature of 4° C. or less before the lymphocytes are disrupted.

77. The method of claim 43, wherein the blood sample and/or the lymphocyte containing sample and/or the plasma containing sample are stored at a temperature of 4° C. or less before the lymphocytes are disrupted.

78. The method of claim 40, wherein the blood sample is whole blood frozen with from about 5% to about 15% DMSO before the lyrnphocytes are disrupted.

79. The method of claim 40, wherein the lymphocytes in said sample are not incubated under conditions, which allow production and/or secretion of antibodies prior to said method.

80. The method of claim 40, wherein multiple samples are tested simultaneously or sequentially.

81. The method of claim 40 for use in identifying an animal infected by an immunogen.

82. The method of claim 81, wherein said animal is a HIV infected patient.

83. The method of claim 81 or 82, wherein the infection of said animal by said immunogen occurs less than 10 days before the blood sample is obtained from said animal.

84. The method of claims 40, for use in high throughput screening.

85. The method of claim 84, wherein said screening is of blood bank blood samples.

86. The method of claim 40, for determining the suitability of a sample for inter-individual transplantation or transfusion.

87. The method of claim 40 for diagnosing or monitoring infection of a human or non-human animal or a part of said animal by an immunogen, wherein the presence or extent of infection is determined by reference to appropriate control and/or reference samples.
